(19) United States
(12) Patent Application Publication
(10) Pub. No.: US 2019/0142291 A1
(43) Pub. Date: May 16, 2019

(54) System and Method for Automatic Interpretation of EEG Signals Using a Deep Learning Statistical Model

(71) Applicant: Temple University-Of The Commonwealth System of Higher Education, Philadelphia, PA (US)

(72) Inventors: Iyad Obeid, Philadelphia, PA (US); Joseph Picone, Elkins Park, PA (US); Amir Hossein Harati Nejad Torbati, Philadelphia, PA (US); Steven D. Tobochnik, Philadelphia, PA (US); Mercedes Jacobson, Philadelphia, PA (US)

(21) Appl. No.: 15/560,658

(22) PCT Filed: Mar. 23, 2016
(86) PCT No.: PCT/US16/23761

§ 371 Date: Sep. 22, 2017

Related U.S. Application Data
(60) Provisional application No. 62/136,934, filed on Mar. 23, 2015.

(51) Int. Cl. A61B 5/04; A61B 5/0476; A61B 5/00; G06N 99/00; G16H 10/60

(52) U.S. Cl. CPC A61B 5/04012; A61B 5/0476; G16H 10/60; A61B 5/7203; G06N 99/005; A61B 5/7267

(57) ABSTRACT

A system and method for automatically interpreting EEG signals is described. In certain aspects, the system and method use a statistical model trained to automatically interpret EEGs using a three-level decision-making process in which event labels are converted into epoch labels. In the first level, the signal is converted to EEG events using a hidden Markov model based system that models the temporal evolution of the signal. In the second level, three stacked denoising autoencoders (SDAs) are implemented with different window sizes to map event labels onto a single composite epoch label vector. In the third level, a probabilistic grammar is applied that combines left and right context with the current label vector to produce a final decision for an epoch. A physician's report with diagnoses, event markers and confidence levels can be generated based on output from the statistical model. Systems and methods for dealing with channel variation or a missing EEG electrode valve are also disclosed. A feature-space boosted maximum mutual information training of discriminative features or an iVectors technique to determine invariant feature components can be implemented for generating a plurality of EEG event labels. An optional GUI allows scrolling by EEG events.

### CROSS-REFERENCE TO RELATED APPLICATIONS

This application is a national stage filing of International Application No. PCT/US16/23761, filed on Mar. 23, 2016, which claims priority to U.S. provisional application No. 62/136,934 filed on Mar. 23, 2015, both of which are incorporated herein by reference in their entireties.

### BACKGROUND OF THE INVENTION

An EEG is used to record the spontaneous electrical activity of the brain over a short period of time, typically 20-40 minutes, by measuring electrical activity along a patient's scalp. In recent years, with the advent of wireless technology, long-term monitoring, occurring over periods of several hours to days has become possible. Ambulatory data collections, in which untethered patients are continuously monitored using wireless communications, are becoming increasingly popular due to their ability to capture seizures and other critical unpredictable events. The signals measured along the scalp can be correlated with brain activity, which makes it a primary tool for diagnosis of brain-related illnesses (see Tatum et al., 2007, Handbook of EEG Interpretation, p. 276; and Yamada et al., 2009, Practical Guide for Clinical Neurophysiologic Testing, p. 416). The electrical signals are digitized and presented in a waveform display. EEG specialists review these waveforms and develop a diagnosis.

EEGs have traditionally been used to diagnose epilepsy and strokes (see Tatum et al.). Other common clinical uses have been for diagnoses of coma, encephalopathies, brain death and sleep disorders. EEGs and other forms of brain imaging such as fMRI are increasingly being used to diagnose head-related trauma injuries, Alzheimer's disease, Posterior Reversible Encephalopathy Syndrome (PRES) and Middle Cerebral Artery Infarction (MCA Infarct). Hence, there is a growing need for expertise to interpret EEGs and, equally important, research to understand how these conditions manifest themselves in the EEG signal.

A board certified EEG specialist currently interprets an EEG. It takes several years of training for a physician to qualify as a clinical specialist. Despite completing a rigorous training process, there is only moderate inter-observer agreement in EEG interpretation (see Van Donselaar et al., 1992, Archives of Neurology, 49(3), 231-237 1992; and Stroink et al., 2006, Developmental Medicine & Child Neurology, 48(5), 374-377).

Machine learning approaches to grand engineering challenges have made tremendous progress over the past three decades due to rapid advances in low-cost highly-parallel computational infrastructure, powerful machine learning algorithms, and, most importantly, big data (Saon et al., 2012). Statistical approaches based on hidden Markov models (HMMs) (Juang and Rabiner, 1991; Picone, 1990) and deep learning (Saon et al., 2015, Proceedings of INTERSPEECH; Hinton et al., 2012, IEEE Signal Processing Magazine, 29(6), 83-97), which can optimize parameters using a closed-loop supervised learning paradigm, have resulted in a new generation of high performance operational systems. Though performance does not yet approach human performance, particularly in noisy conditions, this generation of machine learning technology does deliver high performance on limited tasks. Due primarily to a lack of data resources, these techniques have yet to be applied to a wide range of biomedical applications.

A significant big data resource, known as the TUH EEG Corpus, has recently become available for EEG interpretation (see Harati et al., 2013, Proceedings of INTERSPEECH) creating a unique opportunity to disrupt the market. This resource enables the application of a new generation of machine learning technology based on deep learning. Deep learning technology automatically self-organizes knowledge in a data-driven manner and learns to emulate a physician's decision-making process. The database includes detailed physician reports and patient medical histories which is critical to the application of deep learning. Few biomedical applications have enough research data available to support such technology development.

HMMs are among the most powerful statistical modeling tools available today for signals that have both a time and frequency domain component. For example, a speech signal can be decomposed into an energy and frequency profile in which particular events in the frequency domain can be used to identify the sound spoken. Nevertheless, it took approximately two decades for this technology to mature for applications such as speech recognition. The challenge of interpreting and finding patterns in EEG signal data is very similar to that of speech related projects with a measure of specialization. The biomedical engineering space, however, is so vast and diverse, that no single application can support this type of focused investment. Therefore, what was previously accomplished by handcrafting technology over many years of research must be done in a more automated manner. Deep learning algorithms have recently been revolutionizing fields such as human language technology because they offer the ability to learn in a self-organizing manner (see Hinton et al., 2012), and alleviate the need for meticulous engineering of a system.

HMMs are explicitly parameterized both in their topology (e.g. number of states) and emission distributions (e.g. Gaussian mixtures). Model comparison methods are traditionally used to optimize the number of states and mixture components. These techniques are often referred to as “shallow” models that lack multiple layers of adaptive features. More recently, nonparametric Bayesian methods have shown the ability to self-organize information in a data-driven fashion (see Harati et al., 2013). These systems adapt to the complexity of the data and balance generalization and discrimination. Deep learning systems take this concept one step further and use a fairly generic, hierarchical structure that is trained in an iterative fashion to learn the necessary mappings from a signal to a symbolic representation. Recent advances in training algorithms have overcome barriers that caused previous generations of this technology to get stuck on low-performing sub-optimal solutions (see Seide et al., 2011, Proceedings of INTERSPEECH, p. 437-440).

Another relevant advance that facilitates the development of the technology disclosed herein is the ability to learn parameters of a model in an unsupervised manner. Performance of unsupervised training on vast amounts of data has recently been shown to approach or even exceed supervised training on much less data (see Hinton et al., 2012; and Novotney et al., 2009, Proceedings of the IEEE International Conference of Acoustics, Speech and Signal Processing, p. 4297-4300), giving rise to the notion of big data—learning from vast archives of noisy, poorly transcribed data. For example, early speech recognition systems required intricately transcribed speech data, which is an expensive and time-consuming process to create (often costing thousands of dollars per minute of speech). Previously, no such data existed for EEG interpretation in the quantity required. There has been growing interest in leveraging less precise big data resources to accelerate the technology development process. Unsupervised training techniques are key to exploiting such resources.

There are two fundamental challenges to automatic interpretation of EEG data—feature extraction and event modeling. Feature extraction is a fairly well understood problem, though equally important in its own right. However, the focus of this approach is event modeling. The types of events to be detected manifest themselves in a variety of forms. EEG signals are often processed in terms of features (see Tatum et al.) such as the anterior-posterior gradient, posterior dominant rhythm, and symmetry of the left and right hemispheres. These events have signatures in both the time and frequency domain and at multiple time scales. Hence it makes sense to use a multi-time scale approach for feature extraction (see Adeli et al., 2003, Journal of Neuroscience Methods, 123(1), 69-87). For example, speech recognition systems use a filter bank approach motivated by the human auditory system. EEG systems use a similar type of analysis based on wavelets.

The standard approach to automatic interpretation of EEGs involves a two-level decision-making process in which event labels are converted into epoch labels. These methods usually treat each event independent of the other events (both across channels and time) and apply some form of a voting or fusion technique to produce an epoch label. These approaches are typically based on static classifiers and ignore the time-varying nature of the signal. Though it is straightforward to combine event hypothesis using techniques such as Support Vector Machines or Random Forests, these approaches produce unacceptably high false alarm rates. Further, detection rates for rare events (e.g. spikes) are close to zero, which makes the system unacceptable for clinical use.

A two-level architecture integrates hidden Markov models for sequential decoding of EEG events with deep learning for decision-making based on temporal and spatial context. For purposes of this disclosure, epochs are classified into one of six classes: (1) SPSW: spike and sharp wave, (2) GPED: generalized periodic epileptiform discharge and triphasic waves, (3) PLED: periodic lateralized epileptiform discharge, (4) EYEM: eye blinks and other related movements, (5) ARTF: other general artifacts that can be ignored or classified as background activity, and (6) BCKG: background activity. Spikes tend to occur in short clusters and are local to a particular set of channels. GPEDs and PLEDs also contain spike-like behavior, but demonstrate this behavior over longer periods of time (e.g., minutes). Neurologists use identification of these three events to create diagnoses.

In FIG. 1A, an example of a typical spike is shown. Spikes can be symptomatic of a brain disorder, but that depends heavily on the context in which they occur. The class SPSW represents spikes that occur in isolation. They can typically be observed on multiple channels that correspond to spatially adjacent electrodes. Spikes occur very infrequently in an EEG—less than 1% of the time. This makes them very hard to detect using standard Bayesian approaches to machine learning, because their prior probabilities are so small. A true Bayesian learning process acknowledges that for error rates on the order of 10% to 50%, it is best to ignore the SPSW class altogether since detection of these events is error prone and does not contribute substantially to the overall goal of optimizing the detection accuracy. Until the detection rate on SPSW rises above a lower bound based on random guessing using prior probabilities, the Bayesian perspective is to ignore this class. This observation is significant to the novelty of this disclosure. Accurate SPSW detection is critical to the success of EEG interpretation technology and something state of the art does not currently address properly.

Periodic lateralized epileptiform discharges (PLEDs) are EEG abnormalities consisting of repetitive spike or sharp wave discharges (Dan et al., 2004, Neurophysiology Asia, 9(S1), 107-108). They are focal or lateralized over one hemisphere, which means they typically appear on adjacent channels in an EEG. They recur at fixed time intervals, which is how they can be differentiated from isolated spikes. When present bilaterally and independently, they have been termed BIPLEDs. An example of a PLED is shown in FIG. 1B. PLEDs have most commonly been associated with cerebral infarctions but are also seen in other cerebral diseases such as encephalitis. These are similar to spikes, but occur repeatedly over longer periods of time. To accurately detect PLEDS, a longer-term context must be used.

Generalized periodic epileptiform discharges (GPEDs) are defined as periodic complexes occupying at least 50% of a standard 30-minute EEG, projected over both hemispheres, in a symmetric, diffuse and synchronous manner (although they may be more prominent in a given region, frequently the anterior regions) (Stern, et al., 2005, Atlas of EEG Patterns, Philadelphia, Pa.). The discharges vary in shape, but usually are characterized by spikes or sharp waves of high amplitude. An example of a GPED is shown in FIG. 1C. These are similar to spikes, but occur repeatedly over longer periods of time. GPEDs can only be detected by considering their long-term behavior. A look across multiple epochs to distinguish between the SPSW, PLED and GPED classes is necessary.

The remaining classes are used to accurately model and classify background noise. For example, eye blinks produce isolated spike-like behavior. Events such as eye blinks can be easily confused as a spike by an untrained observer. A typical burst from an eye blink is shown in FIG. 1D. Developing explicit models for artifacts and eye movements improves the ability to differentiate background from the three primary spike-related classes. Separate models can be used for eye movements to improve the ability to detect and ignore artifacts.

A straightforward approach to classifying epochs would be to only use information from the current epoch. However, context plays an important role in these decisions. For example, the spatial location of an event will help determine its classification (e.g., four channels from the front temporal lobe containing a spike event is an indication this is a legitimate spike as opposed to just background noise). Further, the difference between an isolated spike and a recurring set of spikes can be key in determining an epoch is part of a GPED event. In fact, multiple epochs can be a GPED but not an SPSW.

Further, physicians often refer to past behavior of a subject to make decisions about observed changes. One way this is dealt with is through a process of adaptation (see Mak et al., 2005, Speech and Audio Processing, IEEE Transactions on, 13(5), 984-992). The ability of a model to match a specific patient's data can be sharpened by postulating a transformation between the generic subject independent parameters and a specific subject's parameters (see Leggetter et al., 1996, Computer Speech & Language, 9(2), 171-185), and then optimizing this transformation using the same data-driven learning techniques used by the overall system. Current commercial EEG systems do not employ this type of data-driven modeling because they tend to be heuristic in nature. Yet, such adaptation or normalization is clearly used by expert readers in determining if there has been a change in a patient's data.

A comparison of performance for several postprocessing algorithms in terms of the detection rate (DET), false alarm rate (FA), detection rate on spikes and sharp waves (SPSW) and the classification error rate (ERR) is shown in TABLE 1.

TABLE 1

| System | DET | FA | SPSW | ERR |
|---|---|---|---|---|
| 1: Simple Heuristics | 99% | 64% | 99% | 74% |
| 2: Random Forests | 85% | 6% | 0% | 37% |
| 3: Autoencoder | 84% | 4% | 0% | 37% |

The FA rate is the most critical to this disclosure. The goal, is a 95% detection rate and a 5% FA rate. The three standard approaches to forming a decision from event labels are: (1) a simple heuristic mapping that makes decisions based on a predefined order of preference (e.g. SPWS>PLED>GPED>ARTF>EYEM>BCKG); (2) application of a decision tree-based classification approach that uses random forests (see Brieman, 2001, Machine Learning, 45(1), 5-32); and (3) a stacked denoising autoencoder (SDA) that has been successfully used in many deep learning systems (see Bengio et al., 2007; Vincent et al., 2008, Proceedings of the 25^{th} International Conference on Machine Learning, p. 1096-1103, New York, N.Y.). The random forest approach has been successfully used in a variety of machine learning applications. It is a very impressive technique that combines a powerful decision tree classifier with advanced machine learning techniques for training based on cross-validation. Performance of these approaches is respectable since the DET rate is high and the FA rate is low. However, a deeper analysis of these systems shows that they are missing virtually all of the SPSW events. This makes these approaches unsuitable for clinical use. There is no way to adjust the DET and FA rates to achieve an acceptable compromise in performance and maintain good SPSW detection.

Many of the observations provided above regarding the deficiencies of the prior art are significant to the novelty of this disclosure for reasons discussed in further detail in the detailed description of the invention.

What is needed in the art is a high performance deep learning technology that can be applied to the automatic interpretation of EEGs. The system should automatically learn the signal processing techniques and knowledge representations needed to achieve high performance, and produce candidate diagnoses and time-aligned markers that direct physicians to areas of interest in the EEGs. The system should be capable of delivering real-time alerts for efficient long-term monitoring applications such as ambulatory EEGs.

Further, what is needed in the art is a high performance deep learning method and system that implements a wider temporal context to differentiate between spikes and background noise. Techniques such as random forests are capable of learning correlations between channels, and can model temporal context to some extent, but they cannot completely learn the knowledge-based dependencies that neurologists use to make these decisions. A more powerful learning algorithm is required.

### SUMMARY OF THE INVENTION

In one aspect of the invention, the algorithm is trained to automatically interpret EEGs using a three-level decision-making process in which event labels are converted into epoch labels. In the first level, the signal is converted to EEG events using a HMM based system that models the temporal evolution of the signal. In the second level, three stacked denoising autoencoders (SDAs) are implemented with different window sizes to map event labels onto a single composite epoch label vector. In the third level, a probabilistic grammar is applied that combines left and right context with the current label vector to produce a final decision for an epoch. An iterative process is also applied to smooth decisions that terminates when no additional changes are occurring in the final label assignments.

These additional steps in processing are critical to correctly distinguishing between isolated spikes, recurring spikes and background because they exploit the long-term differences between isolated phenomena (e.g., spikes) and recurring phenomena (e.g., periodic spike sequences). While conventional approaches with careful tuning can achieve good detection accuracy and a low false alarm rate, they achieve a very high error rate on spike events. The disclosed three-level system maintains good overall performance yet significantly improves accuracy on spike events.

The system and method described herein can be used to produce a machine-generated interpretation of the EEG and automatically generates a physician's EEG report that includes critical billing information (e.g., ICD codes). Clinical benefits include the regularization of reports, real-time feedback to the patient and decision-making support to physicians. This alleviates the bottleneck of inadequate resources to monitor and interpret these tests.

In one aspect, the invention is a method for automatic interpretation of EEG signals acquired from a patient including the steps of applying the EEG signals to a statistical model, generating multiple EEG event labels, processing the multiple EEG event labels through a first stacked denoising autoencoder including a first window size and configured to map the multiple EEG event labels into one of a first case and a second case, processing the multiple EEG event labels through a second stacked denoising autoencoder including a second window size and configured to map the multiple EEG event labels to one of a first class and a second class, and processing the multiple EEG event labels through a third stacked denoising autoencoder comprising an third window size and configured to map the multiple EEG event labels to one of a complete set of classes, wherein the third window size is longer than each of the first window size and the second window size. The method also includes the steps of generating an output from the statistical model corresponding to the EEG event labels, and generating a report based on the output.

In another aspect, the invention is a system for automatic interpretation of EEG signals including an input component, a memory unit storing a statistical model, and a user feedback device all operably connected to a controller. The statistical model is configured to generate multiple EEG event labels, process the multiple EEG event labels through a first stacked denoising autoencoder comprising a first window size and configured to map the multiple EEG event labels into one of a first case and a second case, process the multiple EEG event labels through a second stacked denoising autoencoder comprising a second window size and configured to map the multiple EEG event labels to one of a first class and a second class, and process the multiple EEG event labels through a third stacked denoising autoencoder comprising a third window size and configured to map the multiple EEG event labels to one of a complete set of classes, wherein the third window size is longer than each of the first window size and the second window size, wherein the statistical model is configured to generate an output corresponding to the EEG event labels, and wherein the system is configured to generate a report based on the output.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIGS. 1A-1D show typical EEGs for common conditions. FIG. 1A is an EEG showing a typical spike. FIG. 1B is an EEG showing periodic lateralized epileptiform discharges (PLEDs). FIG. 1C is an EEG showing generalized periodic epileptiform discharges (GPEDs). FIG. 1D is an EEG showing a typical eye blink.

FIG. 2 is a system for automatically interpreting EEG signals according to an aspect of an embodiment of the invention.

FIG. 3 is an image of an exemplary GUI according to an aspect of an embodiment of the invention.

FIG. 4 is an image of an exemplary physician's EEG report according to an aspect of an embodiment of the invention.

FIG. 5 is a diagram summarizing the statistical model architecture.

FIG. 6 is a diagram showing an architecture for a statistical model for automatically interpreting EEG signals according to an aspect of an embodiment of the invention.

FIG. 7 is a diagram of an iterative hidden Markov model training procedure.

FIG. 8 is a reference map of electrode positions for clinical EEGs.

FIG. 9 is an anatomic diagram of electrode positions for a standard 10/20 EEG.

FIG. 10 is a diagram showing spatial interpolation of EEG signal to reconstruct a missing channel by averaging spatially adjacent channels.

FIG. 11 is a diagram showing a two-level architecture for automatic EEG interpretation.

FIG. 12 is an automatic EEG interpretation system GUI and EEG visualization tool.

### DETAILED DESCRIPTION OF THE INVENTION

The present invention can be understood more readily by reference to the following detailed description, the examples included therein, and to the figures and their following description. The drawings, which are not necessarily to scale, depict selected preferred embodiments and are not intended to limit the scope of the invention. The detailed description illustrates by way of example, not by way of limitation, the principles of the invention. The skilled artisan will readily appreciate that the devices and methods described herein are merely examples and that variations can be made without departing from the spirit and scope of the invention. It is also to be understood that the terminology used herein is for the purpose of describing particular embodiments only and is not intended to be limiting. It is to be understood that the figures and descriptions of the present invention have been simplified to illustrate elements that are relevant for a more clear comprehension of the present invention, while eliminating, for the purpose of clarity, many other elements found in systems and methods of automatically interpreting an EEG. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20%, ±10%, +5%, +1%, and ±0.1% from the specified value, as such variations are appropriate.

“ARTF” as used herein refers to other general artifacts that can be ignored or classified as background activity.

“BCKG” as used herein refers to background activity.

“EEG” as used herein refers to electroencephalography or an electroencephalogram.

“EYEM” as used herein refers to eye blinks and other related movements.

“fBMMI” as used herein refers to feature-space boosted maximum mutual information.

“FFT” as used herein refers to Fast Fourier Transform.

“GPED” as used herein refers to generalized periodic epileptiform discharge and triphasic waves.

“GUI” as used herein refers to a graphical user interface.

“ICA” as used herein refers to independent components analysis.

“MCA Infarct” as used herein refers to Middle Cerebral Artery Infarction.

“MFCC” as used herein refers to mel-frequency cepstral coefficients.

“MLLR” as used herein refers to maximum likelihood linear regression.

“PCA” as used herein refers to principal component analysis.

“PLED” as used herein refers to periodic lateralized epileptiform discharge.

“PRES” as used herein refers to Posterior Reversible Encephalopathy Syndrome.

“RBM” as used herein refers to restricted Boltzmann machines.

“SDA” as used herein refers to stacked denoising autoencoder.

“SPSW” as used herein refers to spike and sharp wave.

“TFRs” as used herein refers to time/frequency representations.

“TUH” as used herein refers to Temple University Hospital.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a system and method for the automatic interpretation of EEG signals.

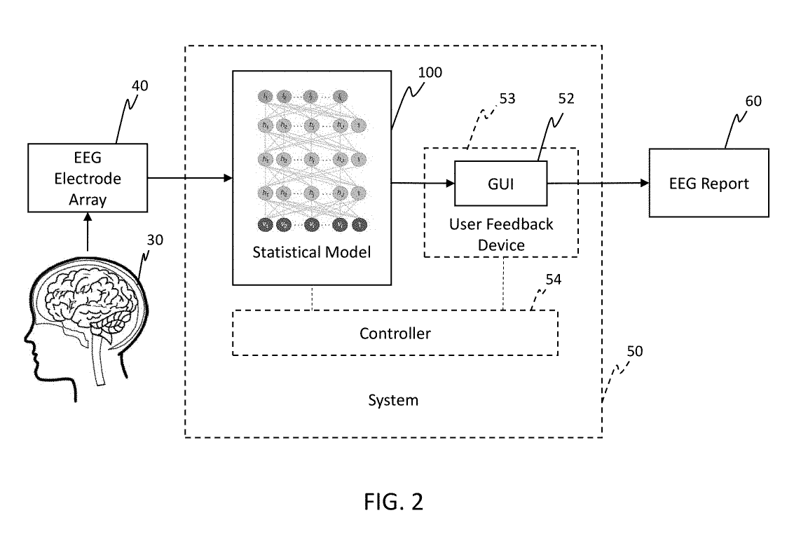

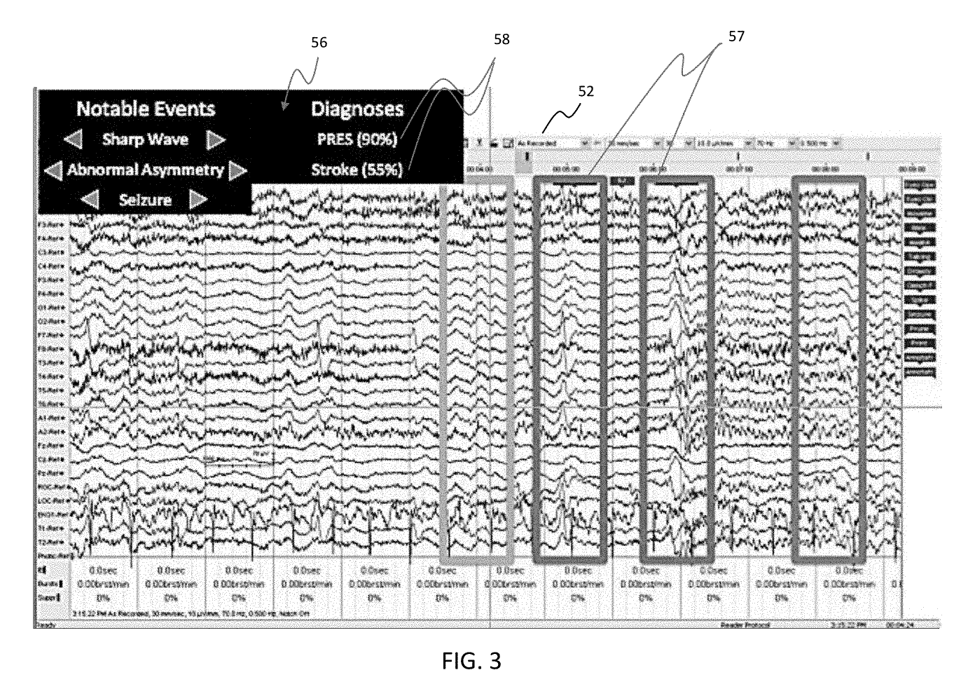

With reference to FIG. 2, an EEG system implementing a trained statistical model 100 is shown according to an exemplary embodiment of the invention. Generally, the system 50 takes EEG measurements recorded from a patient 30 as input, and after the data is processed through the system 50, and more specifically the statistical model 100, a standardized physician's report 60 is generated as output. For acquisition of the EEG signals, an array of EEG electrodes 40 are placed on the scalp of a patient 30. The electrodes 40 are typically either directly attached to the scalp with a conductive gel or paste, or in contact with the scalp by use of an EEG electrode cap or net. Each electrode in the array 40 is connected to an input component operably connected to the system 100. The measured EEG signals can be saved into memory for input and processing at a later time, or directly fed to the system and the statistical model 100 via the input component for real-time processing. The measured EEG signals are processed using a trained statistical model 100. The deep learning algorithm for training the statistical model 100 will be provided in further detail below. Although the statistical model 100 requires massive super computing resources to train, it is extremely run-time efficient and can operate in real-time on modest computing hardware for tasks of this complexity in accordance with the computing systems and architecture described in further detail below. The system 50 can be a readily available computing device, such as a desktop or laptop computer, or a high performance mobile device, such as a high performance tablet. The system includes an integrated controller 54 and memory module (not shown). A GUI 52 can be implemented on a user feedback device 53 such as a touch screen display can be integrated into the system 50, or attached as a separate component. With reference now to FIG. 3, an example of a GUI 52 is shown, demonstrating that physicians can select a diagnosis 56 and be shown the corresponding markers 57. Candidate diagnoses 56 are generated with a confidence level 58 that indicates the system's 50 overall confidence in the prediction. Physicians can navigate by diagnosis, by markers, or simple temporal scrolling. User feedback can also be provided in the form of audio by operably connecting a speaker to the system 50 and controller 54. The system 50 also has a communication unit (not shown) capable of communicating with remote servers, such as cloud-based databases. The communication unit can also use wireless protocols such as Bluetooth for communicating with mobile devices or auxiliary devices such as printers. Wireless computing devices can be used to review the reports, which can also be sent to printers for a hard copy. Either of these communication methods enables medical professionals to monitor patient EEG activity from a remote location. The communication system also allows for the collection of EEG recordings for easily updating the central EEG database.

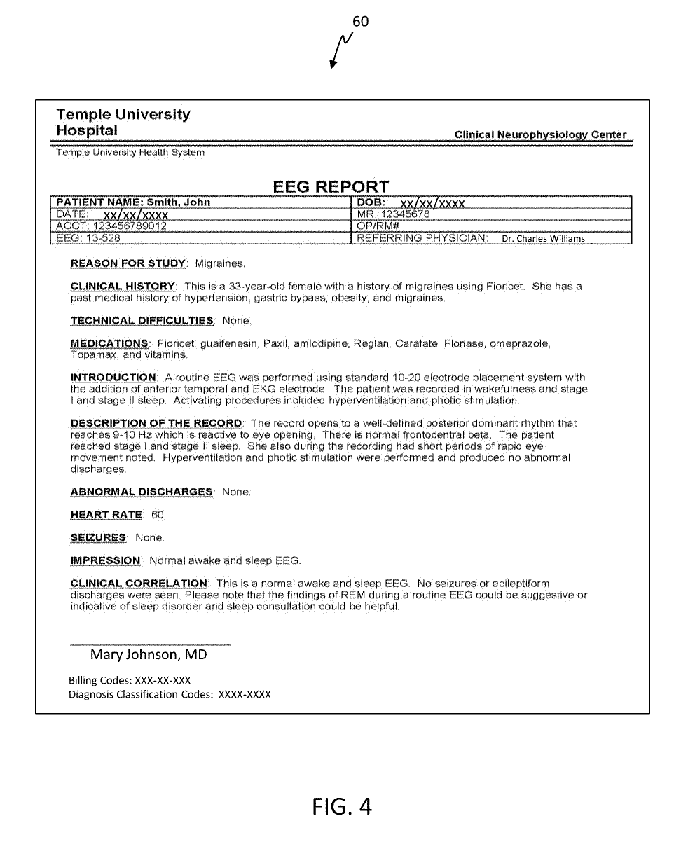

In addition to signal data, for each EEG, a physician's EEG Report 60 is generated based on the output from the statistical model 100. An exemplary embodiment of this report is shown in FIG. 4. The report 60 includes fields that summarize the patient's clinical history and medications. It also includes fields for the physician's findings, which in certain embodiments can be captured in fields called “Impression” and “Clinical Correlation”. This report 60 information is available in an Excel spreadsheet in a name/value pair format. EEGs can also include billing codes and International Classification of Diseases codes (ICD-9). These codes can also form the basis for the classification labels used in machine learning experiments. The system 50 thus provides a uniform and consistent report 60 and format for physicians and health care institutions. Further, fields such as Impression and Clinical Correlation has be used to trigger billing, which provides for a more consistent application of billing schemes. The report 60 can be presented in the GUI 52, and can also be sent via the communications system to a patient database or an auxiliary printer for review and inclusion into the patient's medical file.

As contemplated herein, the present invention includes a system platform for performing and executing the aforementioned methods and algorithms for automatic interpretation of EEG signals. In some embodiments, the EEG system of the present invention may operate on a computer platform, such as a local or remote executable software platform, or as a hosted Internet or network program or portal. In certain embodiments, only portions of the system may be computer operated, or in other embodiments, the entire system may be computer operated. As contemplated herein, any computing device as would be understood by those skilled in the art may be used with the system, including desktop or mobile devices, laptops, desktops, tablets, smartphones or other wireless digital/cellular phones, or other thin client devices as would be understood by those skilled in the art. The platform is fully integrable for use with any additional platform and data output that may be used, for example with the automatic interpretation of EEG signals.

For example, the computer operable component(s) of the EEG system may reside entirely on a single computing device, or may reside on a central server and run on any number of end-user devices via a communications network. The computing devices may include at least one processor, standard input and output devices, as well as all hardware and software typically found on computing devices for storing data and running programs, and for sending and receiving data over a network, if needed. If a central server is used, it may be one server or, more preferably, a combination of scalable servers, providing functionality as a network mainframe server, a web server, a mail server and central database server, all maintained and managed by an administrator or operator of the system. The computing device(s) may also be connected directly or via a network to remote databases, such as for additional storage backup, and to allow for the communication of files, email, software, and any other data formats between two or more computing devices, such as between the system and an EEG database. There are no limitations to the number, type or connectivity of the databases utilized by the system of the present invention. The communications network can be a wide area network and may be any suitable networked system understood by those having ordinary skill in the art, such as, for example, an open, wide area network (e.g., the Internet), an electronic network, an optical network, a wireless network, a physically secure network or virtual private network, and any combinations thereof. The communications network may also include any intermediate nodes, such as gateways, routers, bridges, Internet service provider networks, public-switched telephone networks, proxy servers, firewalls, and the like, such that the communications network may be suitable for the transmission of information items and other data throughout the system.

Further, the communications network may also use standard architecture and protocols as understood by those skilled in the art, such as, for example, a packet switched network for transporting information and packets in accordance with a standard transmission control protocol/Internet protocol (“TCP/IP”). Additionally, the system may utilize any conventional operating platform or combination of platforms (Windows, Mac OS, Unix, Linux, Android, etc.) and may utilize any conventional networking and communications software as would be understood by those skilled in the art.

To protect data, such as sensitive EEG patient information and diagnosis information, and to comply with state and federal healthcare laws, an encryption standard may be used to protect files from unauthorized interception over the network. Any encryption standard or authentication method as may be understood by those having ordinary skill in the art may be used at any point in the system of the present invention. For example, encryption may be accomplished by encrypting an output file by using a Secure Socket Layer (SSL) with dual key encryption. Additionally, the system may limit data manipulation, or information access. For example, a system administrator may allow for administration at one or more levels, such as at an individual reviewer, a review team manager, a quality control review manager, or a system manager. A system administrator may also implement access or use restrictions for users at any level. Such restrictions may include, for example, the assignment of user names and passwords that allow the use of the present invention, or the selection of one or more data types that the subservient user is allowed to view or manipulate.

As described in further detail herein, the EEG system may operate as application software, which may be managed by a local or remote computing device. The software may include a software framework or architecture that optimizes ease of use of at least one existing software platform, and that may also extend the capabilities of at least one existing software platform. The application architecture may approximate the actual way users organize and manage electronic files, and thus may organize use activities in a natural, coherent manner while delivering use activities through a simple, consistent, and intuitive interface within each application and across applications. The architecture may also be reusable, providing plug-in capability to any number of applications, without extensive re-programming, which may enable parties outside of the system to create components that plug into the architecture. Thus, software or portals in the architecture may be extensible and new software or portals may be created for the architecture by any party.

The EEG system may provide software applications accessible to one or more users, such as different users associated with a single healthcare institution, to perform one or more functions. Such applications may be available at the same location as the user, or at a location remote from the user. Each application may provide a graphical user interface (GUI) for ease of interaction by the user with information resident in the system. A GUI may be specific to a user, set of users, or type of user, or may be the same for all users or a selected subset of users. The system software may also provide a master GUI set that allows a user to select or interact with GUIs of one or more other applications, or that allows a user to simultaneously access a variety of information otherwise available through any portion of the system.

The system software may also be a portal or SaaS that provides, via the GUI, remote access to and from the EEG system of the present invention. The software may include, for example, a network browser, as well as other standard applications. The software may also include the ability, either automatically based upon a user request in another application, or by a user request, to search, or otherwise retrieve particular data from one or more remote points, such as on the Internet or from a limited or restricted database. The software may vary by user type, or may be available to only a certain user type, depending on the needs of the system. Users may have some portions, or all of the application software resident on a local computing device, or may simply have linking mechanisms, as understood by those skilled in the art, to link a computing device to the software running on a central server via the communications network, for example. As such, any device having, or having access to, the software may be capable of uploading, or downloading, any information item or data collection item, or informational files to be associated with such files.

Presentation of data through the software may be in any sort and number of selectable formats. For example, a multi-layer format may be used, wherein additional information is available by viewing successively lower layers of presented information. Such layers may be made available by the use of drop down menus, tabbed folder files, or other layering techniques understood by those skilled in the art or through a novel natural language interface as described herein throughout.

The EEG system software may also include standard reporting mechanisms, such as generating a printable EEG results report as described in further detail below, or an electronic results report that can be transmitted to any communicatively connected computing device, such as a generated email message or file attachment. Likewise, particular results of the aforementioned system can trigger an alert signal, such as the generation of an alert email, text or phone call, to alert a medical professional. Further embodiments of such mechanisms are described elsewhere herein or may standard systems understood by those skilled in the art.

Accordingly, the system of the present invention may be used for automatic interpretation of EEG signals. In certain embodiments, the system may include a software platform run on a computing device that provides the EEG diagnosis, waveform, and related information such as applicable billing codes. In one embodiment, the system may include a software platform run on a computing device that performs the deep learning steps described herein.

The algorithm used to automatically interpret EEG signals is a statistical model that is trained automatically, using an underlying machine learning technology and methodology for unsupervised deep learning. The application of this algorithm is in the clinical setting, as part of an EEG system 50 for automated EEG interpretation. The application of such an algorithm generally involves three phases: design, model training and implementation. In the design phase, numbers of inputs and outputs, a number of layers, and the function of nodes are defined. In the training phase, weights of nodes are determined through a deep learning process. Lastly, the statistical model is implemented using the fixed parameters of the network determined during the deep learning phase.

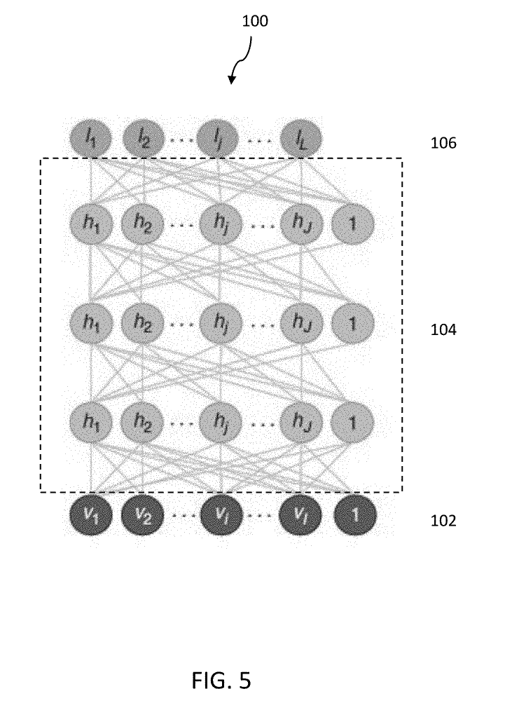

Now with reference to FIG. 5, a summary of the statistical model 100 architecture is shown. The hierarchical system of the statistical model 100 is trained so that through a series of levels or hidden layers 104, it maps features to fundamental units (autonomously learned by the system), and in turn maps these units to outcomes, such as the physician's report 60. The bottom row of states 102, denoted by {vi}, represent the inputs, and the top level of states 106, denoted by {li}, represent the output. In certain embodiments, restricted Boltzmann machines (RBM) are used to implement the hierarchy of networks (see Hinton, 2002, Neural Comput., 14(8) 1771-1800). A RBM consists of a layer of stochastic binary “visible” units that represent binary input data. These are connected to a layer of stochastic binary hidden units that learn to model significant dependencies between the visible units. A RBM can be considered as a type of Markov random field but differs in a number of ways including the fact that it does not usually share weights between different units. In certain embodiments, since EEG data is sequential data, RBMs are combined with conventional HMMs using an architecture where low-level feature extraction and signal modeling is performed using the RBM, and higher-level knowledge processing is performed using some form of a finite state machine or transducer (see Sainath et al., 2012, Acoustics, Speech and Signal Processing (ICASSP), 2012 IEEE International Conference on, 4153-4156).

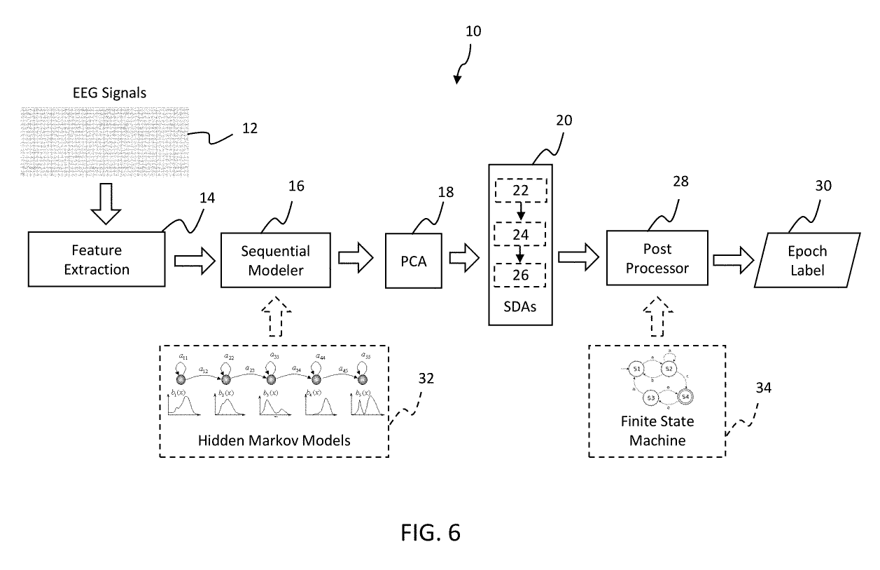

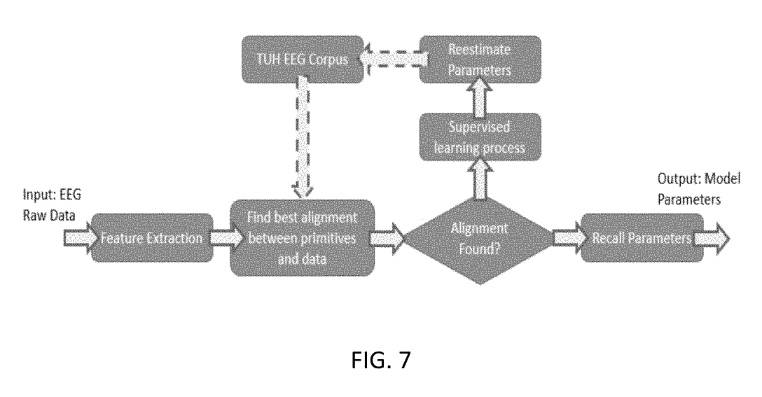

Now with reference to FIG. 6, the statistical model used for processing the EEG signals is trained using a deep learning technique and design that incorporates a variable temporal context with stacked denoising autoencoders (SDAs). Machine learning algorithms are very consumptive of data. These models have millions of degrees of freedom, and need to observe at least one hundred tokens per parameter to reliably estimate its parameters. Powerful computational resources are required to process such data, since the algorithms iterate many times over the data. The EEG signals are acquired 12 and the waveform from individual EEG channels is separated into a number of epochs. Features from each epoch are identified using a feature extraction technique 14 known in the art. The acquired EEG signal is a time domain signal, and features are often hidden among noise in the signal. Features can be extracted using known techniques such as Fast Fourier Transform (FFT) by applying the FFT to the signal and finding its spectrum. In certain embodiments, feature extraction is performed on the data using a standard filter bank/cepstral coefficient approach (see M. Brookes, 1997, “Voicebox: Speech processing toolbox for matlab,” Dept. of Electrical & Electronic Engineering, Imperial College). In the exemplary embodiment, HMMs 18 are combined with RBMs in a sequential modeler 16 for low-level feature extraction and signal modeling. After extracting features, a standard HMM was trained for each class (see L. Rabiner, 1989, Proceedings of the IEEE, vol. 77, no. 2, p. 257-286). HMMs are a class of doubly stochastic processes in which discrete state sequences are modeled as a Markov chain and have been used extensively used to model time series data. An Expectation-Maximization algorithm is used to train the models. An overview of an exemplary iterative HMM training procedure is shown in FIG. 7. An active learning approach is used to bootstrap the system to handle large amounts of data. It should also be noted that data preparation is a large part of the challenge in processing this clinical data. This involves clustering files into the appropriate classes based on information automatically extracted from a physician's report. The system was initially trained in a completely unsupervised manner using an active learning approach. Then, a small amount of data was manually labeled by an expert. 100 10-second epochs were manually selected that contained ample examples of the SPSW class along with a few GPED and PLED examples. This data was used to guide the training process.

With reference back to FIG. 6, and in an exemplary embodiment, the output of the first stage of processing is a vector of six scores, or likelihoods, for each channel at each epoch. Therefore, if have 22 channels and 6 classes we will have a vector of dimension 6×22=132 for each epoch. An event vector for a channel is estimated using a channel-independent model and does not use information from adjacent channels in the same epoch. As recognized by those having ordinary skill in the art, a channel-dependent model could easily be developed. Similarly, the 132-dimension epoch vector is computed without considering similar vectors from epochs adjacent in time. Information available from other channels within the same epoch is referred to as “spatial” context since each channel corresponds to a specific electrode location on the skull. Information available from other epochs is referred to as “temporal” context.

Data is preprocessed using principal component analysis (PCA) 18 to reduce the dimensionality before applying it to these SDAs. PCA 18 is applied to each individual epoch (1 second) for the output of stage 1. In an exemplary embodiment, the input to this process is a vector of dimension 6×22×window length−6 channels times the number of channels in an EEG (there are typically 22 channels of interest in a standard 10/20 EEG configuration) times the number of epochs in the window (e.g., for a 41-second window, this is 41). Hence, the input dimensionality is high—5412. The output of the PCA is a vector of dimension 13 for detectors that look for spikes and eye movements. Three consecutive outputs are averaged, so the output is further reduced from 3×13 to just 13, using a sliding window approach to averaging. The output is 20×window length, or 820, for the detector that chooses between all six classes.

The goal of second and third levels of processing is to integrate spatial and temporal context to improve decision-making. The second stage of processing consists of three stacked denoising autoencoders (SDAs) 20. Each SDA uses a different window size, accounting for a different amount of temporal context. The SDAs map event score vectors onto an epoch label vector, which also contains scores for each class. This mapping is the first step in producing a summary judgment for the epoch based on what channel events have been observed.

These three SDAs 20 improve the performance of the system on rare events (e.g., SPSW). A first SDA 22 is responsible for mapping labels into one of two cases: epileptiform and non-epileptiform. A second SDA 24 maps labels onto the background (BCKG) and eye movement (EYEM) classes. A third SDA 26 maps labels to any one of the six possible classes. The first two SDAs 22, 24 use a relatively short window context because SPSW and EYEM are localized events and can only be detected when we have adequate temporal resolution. In an exemplary embodiment, epochs are restricted to one-second intervals and further subdivide epochs into 100 msec frames used in the hidden Markov model-based event detectors. The first and second SDAs 22, 24 use a three second analysis window weighted such that 90% of the window energy resides at the center of the analysis window.

The third SDA uses a longer window. In an exemplary embodiment, a 41 second uniform window (20 seconds on each side of the center of the window) is used. The length of this window was determined experimentally working with an expert neurologist and analyzing how much context was being used to make local decisions. Neurologists typically view waveforms in 10-second windows, so this longer window essentially provides two windows of context before and after the event under consideration. It was clear from empirical studies that neurologists use more than a 10-second window in making decisions, and hence there is a need to do additional context-based processing. However, decisions about localized events such as SPSW are often made using the limited context described here.

The output of these three SDAs 20 is then combined to obtain the final decision. To add the three outputs together, we initialize our final probability output with the output of the 6-way classifier. For each epoch, if the other two classifiers detect epileptiform or eye movement and the 6-way classifier was not in agreement with this, we update the output probability based on the output of 2-way classifiers. The overall result of the second stage is a probability vector of dimension six containing a likelihood that each label could have occurred in the epoch. It should also be noted that the output of these SDAs are in the form of probability vectors. A soft decision paradigm is used rather hard decisions because this output is smoothed in the third stage of processing.

The results for this system are shown in row 4 of in TABLE 2.

TABLE 2

| System | DET | FA | SPSW | ERR |
|---|---|---|---|---|
| 1: Simple Heuristics | 99% | 64% | 99% | 74% |
| 2: Random Forests | 85% | 6% | 0% | 37% |
| 3: Autoencoder | 84% | 4% | 0% | 37% |
| 4: Postprocessing | 82% | 4% | 42% | 39% |

This system correctly classifies 42% of the spikes and detects another 32% as GPED or PLED. In contrast, our baseline system using random forests, row 2 in TABLE 2, detects 0% of the SPSWs correctly as SPSWS and only detects 30% as GPEDs or PLEDs. The heuristic system, row 1 in TABLE 2, can detect 99% of SPSWs but it also finds a huge number of BCKGs and ARTFs as SPSWs, which makes it clinically useless (a high detection rate can always be achieved when the false alarm rate is also high).

Neurologists generally impose certain restrictions on events when interpreting an EEG. For example, PLEDs and GPEDs don't happen in the same session. None of the first three systems address this problem. The fourth system, introduced above, addresses this consistency issue to some extent, though the final decisions are not strictly constrained to prevent PLEDs and GPEDs from occurring in the final output. In the next section we introduce a third stage that solves this problem and improves the overall detection performance.

The output of the second stage accounts mostly for channel context and is not extremely effective at modeling long-term temporal context. The third stage is designed to impose some contextual restrictions on the output of the second stage. These contextual relationships involve long-term behavior of the signal and are learned in a data-driven fashion. A probabilistic grammar (see Levinson, 2005, Mathematical Models for Speech Technology, p. 119-135) is used that combines the left and right contexts with the labels and updates the labels iteratively until convergence is reached. This is done using a finite state machine that imposes specific syntactic constraints. In an exemplary embodiment, this finite state machine is determined using data-driven training techniques (see Jelinek, 1997, Statistical Methods for Speech Recognition, p. 305). A bigram probabilistic language model that provides the probability of transiting from one type of epoch to another (e.g. PLED*PLED) is trained on a large amount of training data—the TUH EEG Corpus in this case (Harati et al., 2014, Proceedings of the IEEE Signal Processing in Medicine and Biology Symposium, Philadelphia, Pa.). This results in a table of probabilities, shown in TABLE 3, which models all possible transitions from one label to the next.

TABLE 3

| i | j | P(i, j) | j | P(i, j) | j | P(i, j) | j | P(i, j) | j | P(i, j) | j | P(i, j) |
|---|---|---|---|---|---|---|---|---|---|---|---|---|
| SPSW | SPSW | 0.40 | PLED | 0.00 | GPED | 0.00 | EYEM | 0.10 | ARTF | 0.20 | BCKG | 0.30 |
| PLED | SPSW | 0.00 | PLED | 0.90 | GPED | 0.00 | EYEM | 0.00 | ARTF | 0.05 | BCKG | 0.05 |
| GPED | SPSW | 0.00 | PLED | 0.00 | GPED | 0.60 | EYEM | 0.00 | ARTF | 0.20 | BCKG | 0.20 |
| EYEM | SPSW | 0.10 | PLED | 0.00 | GPED | 0.00 | EYEM | 0.40 | ARTF | 0.10 | BCKG | 0.40 |
| ARTF | SPSW | 0.23 | PLED | 0.05 | GPED | 0.05 | EYEM | 0.23 | ARTF | 0.23 | BCKG | 0.23 |
| BCKG | SPSW | 0.33 | PLED | 0.05 | GPED | 0.05 | EYEM | 0.23 | ARTF | 0.13 | BCKG | 0.23 |

The bigram probabilities for each of the six classes are shown. The first column represents the current class. The remaining columns alternate between the class label being transitioned to and its associated probability. The probabilities in this table are optimized on a large training database of transcribed EEG data—in this case the TUH EEG Corpus. For example, since PLEDs are long-term events, the probability of transitioning from one PLED to the next is high—approximately 0.9. However, since spikes that occur in groups are PLEDs or GPEDs, and not SPSW, the probability of transitioning from a PLED to SPSW is 0.0. These transition probabilities emulate the contextual knowledge used by neurologists.

After compiling the probability table, a long window is centered on each epoch and the posterior probability vector for that epoch is updated by considering left and right context as a prior (essentially predicting the current epoch from its left and right context). A Bayesian framework is used to update the probabilities of this grammar for a single iteration of the algorithm:

$${P}_{gprior} = \frac{{\sum }_{i = 1}^{L} {P}_{i} + {ɛ}_{prior} M}{L + M} , \\ L • n umber of epochs in file$$

$$RPP ( k ) = \frac{{β}_{R} {\sum }_{i = 1}^{N} exp ( - i λ ) {P}_{k + i} + {α P}_{gprior}}{1 + α} , \\ Window length = 2 N + 1$$

$$LPP ( k ) = \frac{{β}_{L} {\sum }_{i = 1}^{N} exp ( - i λ ) {P}_{k - i} + {α P}_{gprior}}{1 + α} , \\ Window length = 2 N + 1$$

$${P}_{{C}_{k} \vert LR} = {β}_{C} {{P}_{{C}_{k}} ( {\sum }_{i = 1}^{K} {\sum }_{j = 1}^{K} LPP ( i ) RPP ( j ) Prob ( i , k ) Prob ( k , j ) )}^{\frac{γ}{n}} , \\ k = 1 , 2 , … , K$$

We assume we have K classes (e.g. 6) and the overall length of file in epochs is L. εₚᵣᵢₒᵣ is the prior probability for an epoch (a vector of length K) and M is the weight associated with this assumption. LPP and RPP are left and right context probabilities respectively. λ is the decaying weight for window (e.g. 0), α is the weight associated with P_{gprior} and β_{R} and β_{L} are normalization factors. P_{c}_{ₖ} is the prior probability and P_{c}_{ₖ}_{/LR} is the posterior probability of epoch C for class k given the left and right contexts. y is the grammar weight (e.g. 1), n is the iteration number (starting from 1) and β_{C} is the normalization factor. Prob(i,j) is the probability table shown in Table 2. The algorithm iterates until the label assignments, which are decoded based on a probability vector, converge.

The final output is propagated back to the output of the first stage to update the event probability vectors based on final label probabilities. Performance is summarized in row 5 of TABLE 4.

TABLE 4

| System | DET | FA | SPSW | ERR |
|---|---|---|---|---|
| 1: Simple Heuristics | 99% | 64% | 99% | 74% |
| 2: Random Forests | 85% | 6% | 0% | 37% |
| 3: Autoencoder | 84% | 4% | 0% | 37% |
| 4: Postprocessing Autoencoders | 82% | 4% | 42% | 39% |
| 5: Stochastic Grammar | 89% | 4% | 45% | 36% |

This additional stage of processing raises the detection rate slightly, maintains a good false alarm rate, and increases the accuracy of spike detection, which was its goal. Equally important, the final results have been manually reviewed with neurologists and confirmed that they are consistent with their judgments.

The role of big data to the model training process cannot be overemphasized. However, one issue with past attempts to compile EEG big data is that the vast majority of EEGs collected at any single institution exhibit normal behavior. For example, at one hospital, there were approximately 21 cases of PRES diagnosed out of 14,000 patients seen in the past 12 years. Obviously, with such lopsided statistics, a small database of several hundred samples, unless carefully constructed to contain a variety of data, will not contain an adequately rich dataset for training. The machine learning algorithms will simply ignore the pathological data and tend to classify everything as normal. Most technology development has been done on such small databases, necessitating the use of heuristic measures. The availability of the TUH EEG Corpus (see Harati et al., 2013) is central to both the technology development and evaluation in this project. The TUH EEG Corpus makes this type of data-driven approach feasible for the first time.

A system that automatically interprets EEGs must somehow map these unique configurations onto a common set of channels in order for typical machine learning technology to be successful. Channel mismatches are notoriously problematic for machine learning. The mapping process typically involves two steps: (1) inverting a montage representation (see ACNS, 2006, Guideline 6: A Proposal for Standard Montages to Be Used in Clinical EEG, 1-7) if the data is not stored as raw channel data and (2) interpolating channels to produce an estimate of a missing electrode. The former, montage inversion, is relatively straightforward and involves simple algebraic manipulations since montages are most often simply differences between a channel (e.g., electrode F1) and a designated reference point on the body (e.g., electrode O2). The latter, interpolation, has historically been done using a simple spatial interpolation process (see Law et al., 1993, IEEE Transactions on Biomedical Engineering, 40(2), 145-153). This is essentially an averaging process that is well known to produce relatively minor improvements in the signal to noise ratio (see van Trees, 2002, Detection, Estimation, and Modulation Theory, Optimum Array Processing (Part IV), 1472).

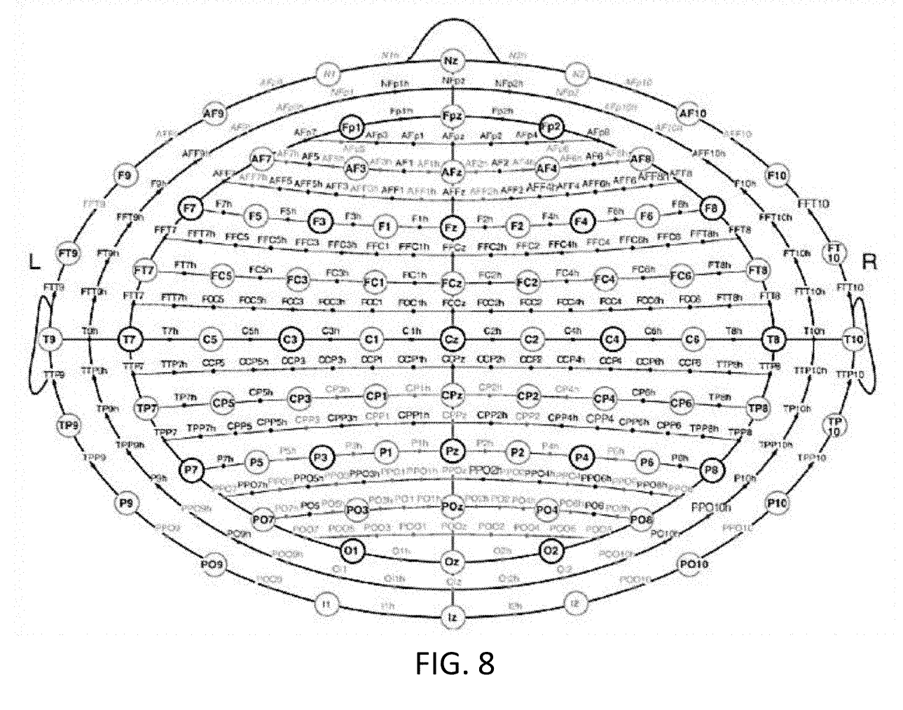

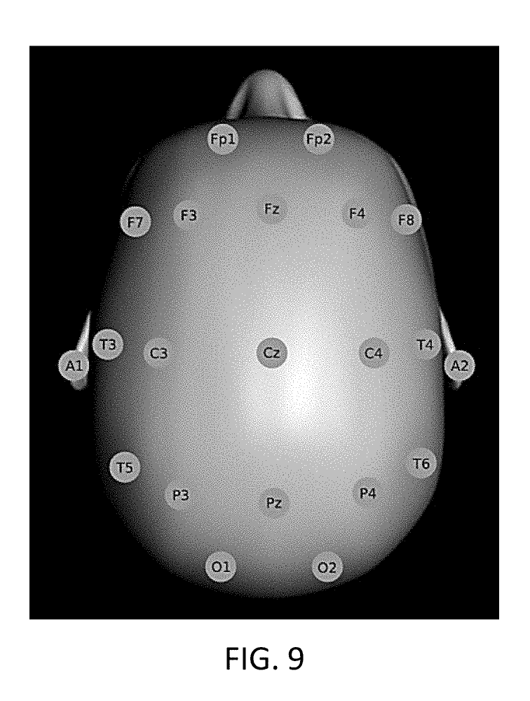

In certain embodiments, the approach to automated interpretation of EEGs includes a step to map all configurations onto a standard 10/20 baseline configuration, which is then converted to a montage that improves the ability to detect spike events. A reference map of electrode positions for clinical EEGs is shown in FIG. 8, with dark circles indicating the position that correspond to a 10/20 configuration. FIG. 9 shows an anatomic diagram of electrode positions for a standard 10/20 EEG. In one aspect, a preprocessor converts an arbitrary EEG multichannel configuration to a standard 10/20 configuration and reconstructs missing channels. To reconstruct a missing channel, an approach based on information theoretic measures such as mutual information, maximum likelihood and linear filtering is implemented. These approaches provide higher performance because they preserve statistically meaningful data in the signal rather than simply minimize mean-squared error. The resulting signal is richer in information and better suited to the needs of subsequent stages of machine learning-based interpretation. This approach is computationally efficient yet is very robust to noise and other artifacts that often appear in these records.

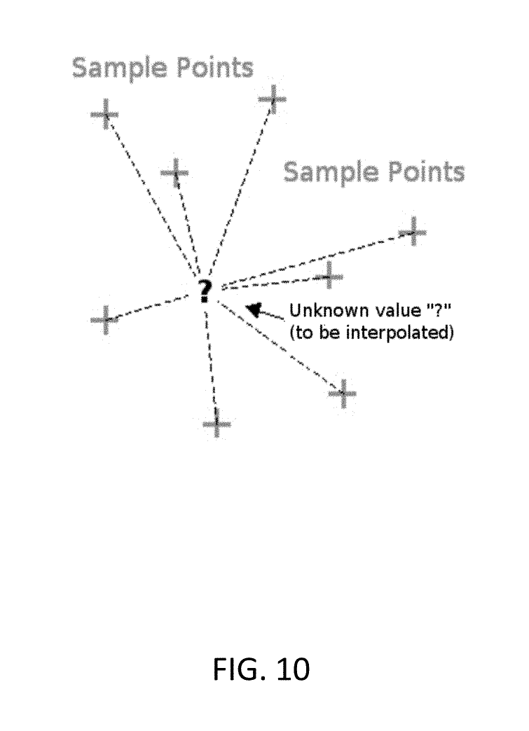

A typical approach to spatial interpolation is shown in FIG. 10. The EEG signal is a multichannel signal which we can denote as x[m,n], where m represents the electrode index and n represents the time index of a sample for that electrode. The interpolated channel can be computed by averaging spatially adjacent channels:

$$x [ p , n ] = ( 1 / M ) {\sum }_{m = 1}^{M} x [ m , n ]$$

where p represents the index of the channel to be interpolated. Historically, averaging is the first and most straightforward technique used since it is based on a well-established theory of array processing (see Johnson, 1993, Array Signal Processing: Concepts and Techniques, 512) and has been successfully employed for many years in audio processing.

More recently, techniques based on mutual information and other information theoretic techniques have emerged. A commonly used nonlinear approach based on mutual information that has been applied to EEG processing is Independent Components Analysis (ICA) (see Makeig, et al., 1996, Advances in Neural Information Processing Systems, 145-151). The most popular form of ICA constructs an estimate of the signal by minimizing mutual information between the adjacent channels. One of its main benefits is the reduction of spurious artifacts in the signal. Head-related transfer functions have also been used to construct 3D images of the head, which can also be used to interpolate to reconstruct missing channels (see Brunet, et al., 2011, Computational Intelligence and Neuroscience). However, these techniques have produced modest results on actual clinical data and are not actively used in clinical settings.

An alternative approach to channel reconstruction is to hypothesize a linear mapping between the input channels and the reconstructed channels, and to optimize this mapping as part of the training process. This technique was initially introduced as Maximum Likelihood Linear Regression (MLLR) (see Leggetter, et al., 1995, Computer Speech & Language, 9(2), 171-185), and subsequently expanded to allow several different styles of training (see Gunawadana et al., 2001, Proceedings of Eurospeech, 1-4; and Harati, et al., 2012, Proceedings of the IEEE International Conference on Acoustics, Speech and Signal Processing, 4321-4324). In certain embodiments, the preference is to employ such methods in the feature space operating on feature vectors since this more directly models important frequency domain phenomena and better integrates with the classification system.

In this method, a linear mapping is hypothesized between the measured channels, vᵢ, and the missing channel, yᵢ:

y[n]=Av[n]

The feature vectors corresponding to frame n, each of which is of dimension p, are concatenated into a supervector, v[n]:

v[n]=[v₁[n]|v₂[n]| . . . |v_{q}[n]]^{T}

where vᵢ[n] is a p-dimensional feature vector corresponding to the i^{th} channel for frame n. The supervector v[n] is of dimension p×q where q is the number of channels.

The transformation matrix A is of dimension p rows and p×q columns. The product of A and the supervector v[n] produces the estimate of the corresponding feature vector for the reconstructed channel, y[n]. Without loss of generality, a constant term can be added to the representation to account for a translation in addition to a multidimensional scaling.

The matrix A represents in general an affine transformation that postulates a linear filtering model describing how to transform the spatially adjacent channels, vᵢ, into a reconstructed channel. There is ample neuroscience evidence to suggest that a linear model should be sufficient to describe this transformation, which is the result of electrical signals being conducted through the scalp. Since the distances between the actual sensors and the missing sensor tend to be small, a piecewise linear spatial model is sufficient.

The parameters of this model are estimated using a closed-loop unsupervised training process identical to what is used in MLLR. The parameters are adjusted to optimize the overall likelihood of the data given the model. Typically, only a small number of iterations of training are required (e.g., three) to reach convergence. As in MLLR, multiple transformation matrices can be hypothesized using a regression tree or nonparametric Bayesian clustering approach (see Harati, et al., 2012). Parameters of this model can also be training using discriminative training or any other type of convex optimization.

The model also can be extended to incorporate temporal context. Features vectors from the previous and future frames in time can be added to the supervector representation. In certain embodiments, a single transformation matrix is adequate and additional temporal context is not needed because the propagation delays between sensors are negligible.

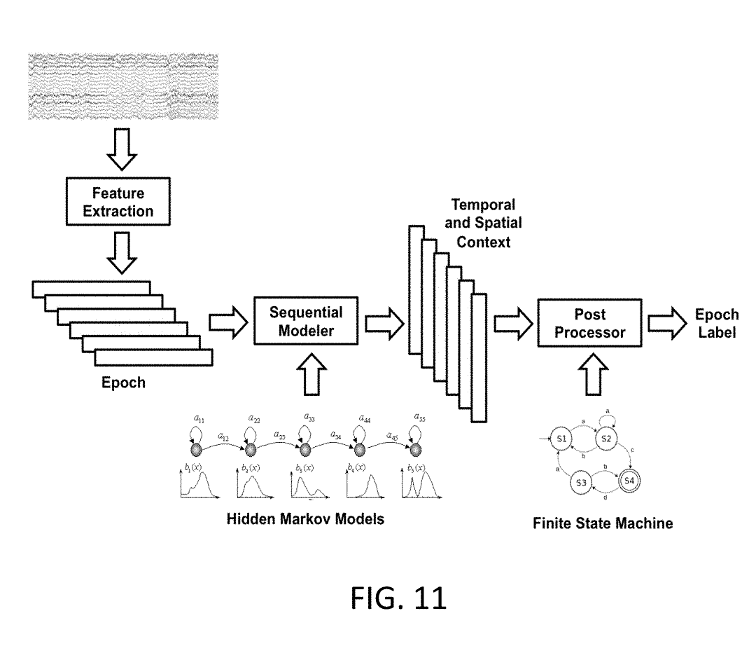

A block diagram of an exemplary overall EEG interpretation system is shown in FIG. 11. The system uses a two-level architecture that integrates principles of hidden Markov models with deep learning. A multichannel EEG signal is input to the system. In certain embodiments, the input can be in the form of a European Data Format (EDF) file. As already discussed above, the EEG signal is a multichannel signal that can contain as few as 3 channels and as many as 128 or 256 channels sampled at or close to 250 Hz and typically represented using 16-bit samples. The signal must be converted to a sequence of feature vectors so that typical machine learning technology can be applied to do EEG event classification. In certain embodiments, features are computed every 100 msec, which is referred to as the frame duration. The output of this stage of the processing is a sequence of vectors containing energy (computed in the frequency domain) and 12 cepstral coefficients. These frames are grouped into an epoch, which consists of 10 frames, or 1 second of data, and passed to the sequential modeler. The system is not restricted to this set of parameters, as this is merely an exemplary embodiment.

Many neurologists prefer a crude form of preprocessing of the signal in which differences between channels are computed and displayed. This is referred to as a montage (ACNS, 2006). For example, when examining an EEG for events that can lead to a diagnosis of epilepsy, a transverse central parietal (TCP) montage is preferred because it accentuates spike behavior. These montages can be regarded as a simplistic form of signal preprocessing before feature extraction. In theory, they can be improved or eliminated completely by a more sophisticated form of feature extraction that uses both spatial and temporal context. Advantageously, a general feature extraction approach is achieved that includes such capabilities. Similar types of approaches have been successfully applied to other forms of signal processing (see Bocchieri et al., 1986, IEEE Transactions on Acoustics, Speech and Language Processing, 34(4), 755-764) but have yet to be applied to EEG clinical data.

Similarly, in many standard feature extraction approaches, absolute features, referred to as features that directly measure attributes of the signal such as the spectrum, can be combined with first and second derivatives, which incorporate temporal behavior of the signal (Picone, 1993, Proceedings of the IEEE, 81(9), 1215-1247). This concatenated feature vector is a useful input into sequential modeling techniques such as hidden Markov models because the feature vector encodes both static and dynamic information about the signal.

Features are crucial to any pattern recognition system. Features must accurately convey meaningful differences between the signals representing various events to be recognized. For example, spikes and sharp waves are an important part of the process that neurologists use to interpret an EEG. Their presence as an isolated event or repetitive event is the basis for determining pathologies such as epilepsy and stroke. Current EEG systems primarily use time domain measures, such as peak/valley ratios measured directly from the EEG signal, to characterize such events. Such measures are notoriously noisy and unreliable, causing excessive amounts of false alarms. As a result, neurologists ignore these advanced analytics in clinical practice. The focus here is to replace such measures with robust and reliable features that exploit both the time and frequency domain properties of the signals.

Signals that display temporal structure that occurs over both short and long time intervals can be analyzed using a technique known as multi-time scale analysis. The most straightforward example of this is the filter bank used in the mel-frequency cepstral coefficients (MFCC) front end (see Davis et al., 1980, IEEE Transactions on Acoustics, Speech, and Signal Processing, 28(4), 357-366). A single channel of the EEG signal is converted to a series of bandpass filtered signals using a linearly or logarithmically spaced filter bank. The subsequent signals are converted to a vector of measurements by periodically computing the energy output from each of these filters, and then enhancing the information contained in these measurements by computing the cepstrum of these values using an inverse discrete cosine transform.

A generalization of this approach that has been utilized in other signal processing applications replaces the filter bank analysis with a wavelet transformation (see Adeli et al., 2003, Journal of Neuroscience Methods, 123(1), 69-87). Wavelets in theory alleviate the need for a discrete filter bank because they produce a true time/frequency representation of the signal. In practice, however, they are implemented in such a way that they produce a result very similar to the MFCC representation, and hence have not delivered significant improvements in performance over the MFCC approach (see Muller, 2007, Speaker Classification I: Fundamentals, Features, and Methods (p. 355)).

Wavelets are just one of many time/frequency representations (TFRs). Perhaps the simplest of these is the spectrogram, which displays the magnitude of the Fourier transform as a function of both time and frequency. This is from a class of time/frequency representations known as linear TFRs. The resolution of this display is controlled at the rate at which the analysis is updated in the time domain (the frame duration) and the amount of data used to compute the spectrum (the window duration). A generalization of the spectrogram is a formulation in which the signal is correlated with itself, often referred to as an autocoherence function. Such representations are known as quadratic TFRs (see Hlawatsch et al., 1992, Linear and quadratic time-frequency signal representations, IEEE Signal Processing Magazine) because the representation is quadratic in the signal. The Wigner-Ville distribution is a well-known example of this.

For many years, research focused on searching for the ultimate set of features using some a priori defined transformation. However, as machine learning advanced, it became clear that even the feature extraction process could benefit from many of the advanced statistical training techniques used in the pattern recognition system. Soon, even feature extraction could be optimized using discriminative training techniques. One such popular approach to such feature generation is known as feature-space boosted maximum mutual information (fBMMI) training of discriminative features (see Povey et al., 2008, Proceedings of the IEEE International Conference on Acoustics, Speech and Signal Processing. Las Vegas, Nev., USA). In this approach the classification error rate is essentially minimized by optimizing a transformation of the feature vectors. This approach is attractive because it has been shown to work well with deep learning based systems (see Rath et al., 2013, Proceedings of INTERSPEECH, 109-113).

Finally, a new technique known as iVectors that is based on the integration of a number of these concepts has emerged (see Dehak et al., 2011, IEEE Transactions on Audio, Speech, and Language Processing, 19(4), 788-798). In this approach, noisy spectral measurements are deconvolved by estimating subject-dependent and channel-dependent components, which in turn reveal the invariant components of the features most useful for classification. A generalized feature extraction software toolkit has been developed that allows implementation of many of these techniques within a uniform framework so that direct comparisons between these techniques can be made. This software allows optimization of features for particular tasks (e.g., spike detection versus historical searches) and real-time performance. Montage generation and feature extraction are specified from a common recipe file that is loaded at run-time and does not require recompilation of the code. Feature extraction runs hyper real time requiring only about 5% of the total computation time required for high performance classification. The system can be configured to operate in a standard single-channel mode as well as modes in which both temporal and spatial context can be incorporated.

Using a straightforward MFCC-based feature extraction process, baseline results have been established on the TUH EEG Corpus of 90% detection accuracy at a false alarm rate of less than 5%. Several of the techniques described above, including fBMMI and iVectors, have yet to be applied to EEG processing. Features based on TFR representations can be added that should increase performance to 92% detection accuracy. Discriminatively trained features can also be added to further increase performance to 95% detection accuracy and reduce the false alarm rate to 2.5%.

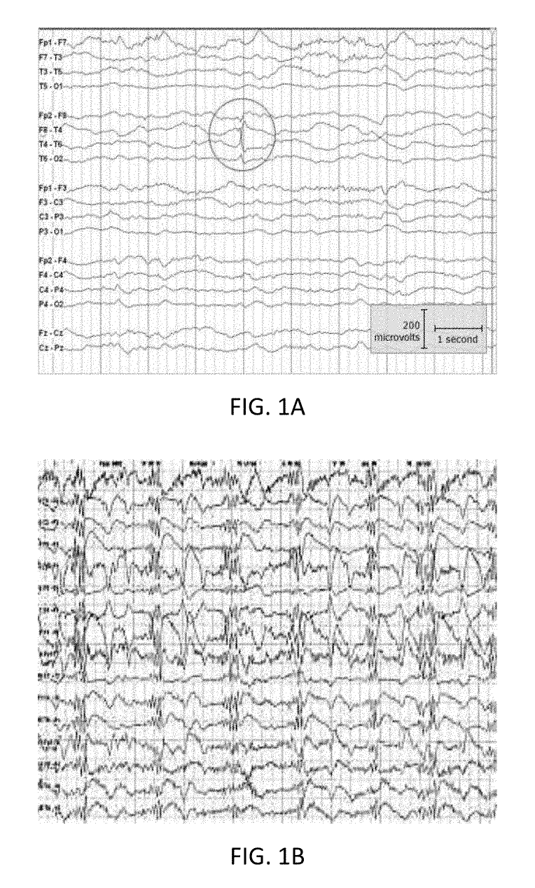

Regarding the user interface, prior to the use of computer technology, EEGs were primary read by reviewing hardcopies from strip chart type displays (see Sanei et al., 2008, EEG signal processing, 312). The craft for interpreting an EEG was developed in this context, and clinicians still relate to the data using this very familiar type of display. A typical waveform display from a computer-based EEG system is shown in FIGS. 1A-1D. These display tools are designed to emulate the look of an EEG printed on paper (e.g., black waveform on a white background).

Perhaps the three most important features of these displays are (1) the implementation of a montage (ACNS, 2006), which specifies a series of differential signals (e.g., T3-T1 implies subtracting channel T1 from channel T3) and the order in which channels are viewed; (2) filtering options which smooth the signals (e.g., apply notch filters to remove line noise and other low frequency artifacts); and (3) the amplitude scale adjustments which allow clinicians to view events on a familiar amplitude scale (e.g., 100 μvolts/mm). Neurologists also prefer to view the waveforms in 10 sec intervals—the number of seconds of the signal per display window (referred to as the page time). They will often measure distances between events using this time scale and are comfortable with this amount of temporal resolution.

To put this in perspective, a clinician would need to page through 6 pages/min.×60 mins./hr.×24 hrs.=8640 displays to read a 24-hour long term monitoring (LTM) EEG. Even if they were able to process one page per second, it would take more than two hours to review such an EEG. Hence, neurologists must scroll through these waveform displays very quickly to keep up with the data being generated, increasing the potential for missing key events in the EEG. Reading of an EEG is an important step in the billing cycle for a patient visit, so delays in reading EEGs translate to delays in billing. Neurologists, of course, would prefer to be seeing patients (and generating revenue) rather than spending time reading and reporting on EEGs. EEGs are often read after hours when neurologists are not seeing patients, further complicating an already packed schedule.

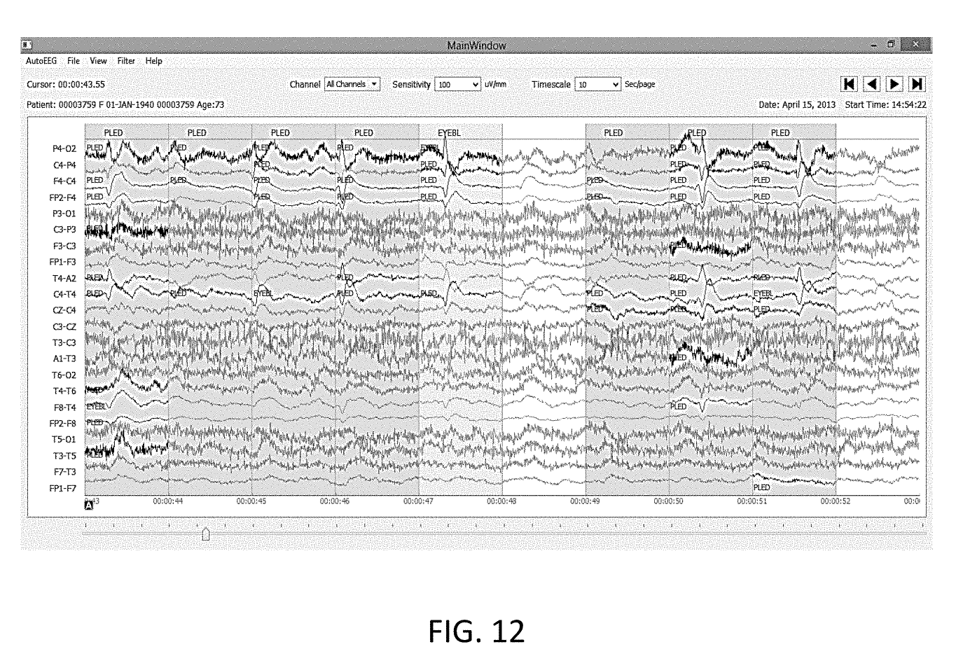

These points are particularly relevant to the novelty of embodiments of the visualization tool and GUI described herein, according to an aspect of the invention. A visualization tool or GUI of an EEG (shown in FIG. 12) has been developed that incorporates a number of new features designed to improve the efficiency of the process of manually interpreting an EEG and enhance the accuracy of these interpretations. The multichannel signal is displayed in a manner similar to FIGS. 1A-1D. Users have access to similar interface options such as paging forward and backward, controlling channel selections, scales, etc. Real-time cursors are provided so that localization of events can be easily documented. The software is implemented so that it can be easily ported to virtually any platform including laptops, tablets and smartphones. Python is used for this in certain embodiments, though any language that is supported across all these devices would be adequate.

One major advantage of the system and GUI disclosed herein is that in certain embodiments it supports paging forward and backward by epoch labels. In FIG. 12, the output of the automatic interpretation system is shown in the form of labels that appear above each channel and above the overall waveform. For example, the grayish areas of the signal show the label “PLED” above each channel indicating that the signal at that point in time has been classified as a PLED event. PLED also appears along the top of the waveform, indicating that the overall assessment of the epoch (typically a one-second interval) was PLED.

The page forward and backward functions allow the user to page forward by event. Similarly, users can search forward or backward by event. This provides clinicians with the ability to focus on specific events of interest, such as a PLED event, and ignore the vast majority of the signal that has no significant abnormalities. This results in an enormous productivity increase. Such a feature is simply not possible without leveraging high performance automatic interpretation technology.

Another major advantage of the system and GUI in certain embodiments is the ability to locate a patient or an EEG with similar characteristics to the EEG being viewed. Users can search a large database of indexed EEGs for relevant patient information. Searchable information may include for example a patient's demographics (e.g., age, date of exam, name, medical record number) and medical history (e.g., medications, previous diagnoses).

Yet another major difference in the system and GUI in certain embodiments is the ability to locate a similar patient based on their pathology. Because the EEGs are automatically labeled and classified, the entire EEG record, including the signal, is searchable. Clinicians can search for patients with similar diseases (e.g., “find all patients that suffer from PRES”) or for patients with similar signal characteristics. This last feature, which has been pioneered in applications like music processing (Kumar et al., 2012, IEEE 14th International Workshop on Multimedia Signal Processing (MMSP). Banff, Canada), allows clinicians to select a section of the signal and find another EEG session that has a similar temporal and spectral characteristic to the selected signal.

Medical students can use this feature to conduct studies into what an event might look like when viewed across multiple sessions. Clinicians can use this feature to compare recent events to previous events for the same or different patients. It is both a training and validation tool.

A final advantageous feature of the visualization tool in certain embodiments is the ability to examine events in both the time domain, which is the current preferred method for reading EEGs, and the frequency domain using a variety of time frequency representations (e.g. a spectrogram). Some events are much easier to discern in the frequency domain or using a combination of temporal and frequency domain queues. The system and GUI tool allows clinicians to seamlessly move between the two domains. The use of a frequency domain display will greatly impact their ability to quickly spot spike and sharp wave events.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

1. A method for automatic interpretation of EEG signals acquired from a patient, the method comprising:
applying the EEG signals to a statistical model;
generating a plurality of EEG event labels based on the EEG signals;
processing the plurality of EEG event labels through a first stacked denoising autoencoder comprising a first window size and configured to map the plurality of EEG event labels into one of a first case and a second case;
processing the plurality of EEG event labels through a second stacked denoising autoencoder comprising a second window size and configured to map the plurality of EEG event labels to one of a first class and a second class;
processing the plurality of EEG event labels through a third stacked denoising autoencoder comprising an third window size and configured to map the plurality of EEG event labels to one of a complete set of classes, wherein the third window size is longer than each of the first window size and the second window size;
generating an output from the statistical model corresponding to the EEG event labels; and
generating a report based on the output.

2. The method of claim 1, wherein the first case is epileptiform and the second case is non-epileptiform.

3. The method of claim 1, wherein the first class is (SPSW) spike and sharp wave and the second class is (EYEM) eye blinks and other related movements.

4. The method of claim 1, wherein the complete set of classes comprises at least four classes.

5. The method of claim 4, wherein the complete set of classes comprises the classes (SPSW) spike and sharp wave, (GPED) generalized periodic epileptiform discharge and triphasic waves, (PLED) periodic lateralized epileptiform discharge, (EYEM) eye blinks and other related movements, (ARTF) other general artifacts that can be ignored or classified as background activity, and (BCKG) background activity.

6. The method of claim 1, wherein at least one of the first window size and the second window size is between 2 seconds and 4 seconds.

7. The method of claim 1, wherein each of the first window size and the second window size is approximately 3 seconds.

8. The method of claim 1, wherein the third window size is between 26 and 56 seconds.

9. The method of claim 1, wherein the third window size is approximately 41 seconds.

10. The method of claim 1 further comprising:
separating a plurality of EEG signals into a plurality of epochs, and extracting features from the plurality of epochs.

11. The method of claim 1 further comprising:
training a plurality of hidden Markov models, wherein each hidden Markov model corresponds to an EEG class.

12. The method of claim 11, wherein EEG signals are converted to EEG event labels based on the hidden Markov models.

13. The method of claim 1 further comprising:
preprocessing EEG event label data using principal component analysis prior to the step of processing through the first stacked denoising autoencoder.

14. The method of claim 1, wherein a graphical user interface is displayed on an interactive user feedback device, the graphical user interface comprising a diagnosis and a corresponding EEG waveform marker based on the output.

15. The method of claim 14, wherein the diagnosis comprises a confidence level.

16. The method of claim 14, wherein the graphical user interface is configured for temporal scrolling of EEG waveforms.

17. The method of claim 1, wherein the report is displayed in a graphical user interface.

18. (canceled)

19. The method of claim 1, wherein the report comprises a diagnosis and a marked portion of an EEG waveform based on the output.

20. (canceled)

21. (canceled)

22. The method of claim 1 further comprising:
processing EEG event labels through a bigram probabilistic language model comprising probabilities of transitioning from one type of epoch to another.

23. The method of claim 22, wherein the complete set of classes comprises the classes (SPSW) spike and sharp wave, (GPED) generalized periodic epileptiform discharge and triphasic waves, (PLED) periodic lateralized epileptiform discharge, (EYEM) eye blinks and other related movements, (ARTF) other general artifacts that can be ignored or classified as background activity, and (BCKG) background activity.

24-58. (canceled)
